(19) United States
(12) Patent Application Publication
(10) Pub. No.: US 2004/0052851 A1
(43) Pub. Date: Mar. 18, 2004

(54) Modified release oral dosage form

(76) Inventors: Allan H. Graff, Parsippany, NJ (US); Stuart J. Platcow, Parsippany, NJ (US)

Correspondence Address: GLAXOSMITHKLINE, Corporate Intellectual Property- UW2220, P.O. Box 1539, King of Prussia, PA, 19406-0939, US

(21) Appl. No.: 10/244,782

(22) Filed: Sep. 16, 2002

(51) Int. Cl. A61K 9/68; A61K 7/20; A61K 9/14

(52) U.S. Cl. 424/488; 424/48; 424/53

(57) ABSTRACT

The present invention is directed to glassy matrix solid oral dosage forms useful for transmucosal oral administration of an active, such as nicotine.

### FIELD OF THE INVENTION

The present invention relates to solid, oral dosage forms comprising an active, e.g., nicotine, which are useful where modified release of the active to the oral mucosa is desired. The invention more particularly concerns such nicotine-containing dosage forms that are useful for reducing or preventing nicotine cravings by oral transmucosal delivery of the nicotine active. The invention also relates to methods of using such compositions for reducing or preventing nicotine cravings or tobacco usage.

### BACKGROUND OF THE INVENTION

It is generally known that active as well as passive smoking of tobacco products, such as cigarettes, cigars, and pipe tobacco, presents serious health risks to the user and those subjected to secondary smoke. It is also known that use of other forms of tobacco, such as chewing tobacco, presents serious health risks to the user. Furthermore, the use of tobacco products in public areas is increasingly either restricted or socially unacceptable.

It is also recognized that reducing or quitting tobacco use is often very difficult for persons accustomed to using tobacco. This difficulty arises in large part from the addictive nature of nicotine. Efforts have therefore been made to provide nicotine substitutes to satisfy a tobacco user's cravings, but which avoid health risks associated with tobacco use, especially smoking.

In recent years, nicotine replacement therapies (NRT) have been successfully commercialized as a means to reduce or quit smoking or other forms of tobacco usage. Such commercial NRT include nicotine gums (e.g., NICORETTE) and nicotine transdermal patches (e.g., NICODERM). While such means are useful as aids to reduce or quit smoking, there is an ongoing need to provide improved or alternate NRT. For example, users may prefer to use forms other than chewing gum or transdermal patches. Certain users may dislike or be unable to chew gum, and users may desire more rapid craving relief than typically provided by transdermal patches.

In addition, nicotine lozenges have been marketed outside of the United States, for example, as STOPPERS and NICOTINELL brand lozenges. As far as the present inventors are aware, such lozenges are in the form of compressed tablets. In addition, U.S. Pat. Nos. 5,593,684; 5,721,257 and 5,362,496 (Baker et al.) disclose methods and therapeutic systems for smoking cessation, utilizing transdermal nicotine delivery for obtaining base-line nicotine plasma levels, coupled with transmucosal administration of nicotine to satisfy transient craving. One preferred transmucosal delivery system is a lozenge for buccal delivery, comprising nicotine dispersed in an absorbent excipient and a nonnutritive sweetener, preferably made by direct compression.

While providing a potential alternate NRT form, such compressed lozenges may not be appealing to certain users for performance or esthetic reasons. For example, compressed tablets tend to have a relatively grainy texture. In addition, commercial tablets of which the present inventors are aware are designed to have a relatively long dissolution period, such that craving relief is not as rapid as might be desired.

Nicotine confectionary forms are disclosed in U.S. Pat. Nos. 6,082,368 (Brown) and 5,048,544 (Mascarelli et al.). Brown discloses a nicotine candy in a cigarette shaped package. The candy may use beta-pyridyl-alpha-N-methyl pyrrolidine or powdered tobacco leaves dissolved or dispersed in any standard hard sugar candy. Examples of sugars for making the hard candy include corn sugar, table sugar, and the sugar-free substitute, Lycasin. Mascarelli et al. discloses a cigarette substitute having an edible portion with nicotine, e.g., in the form of a conventional lollypop preferably with a hard or semi-hard candy.

Copending PCT International Application No. PCT/US02/08914, filed Mar. 22, 2002, entitled “Nicotine-Containing Oral Dosage Form” (C75119), discloses a hard-boiled oral dosage form for nicotine which is free water soluble gelling agents, e.g., gums, and which comprises a glassy matrix of a sugar alcohol, or a mixture of sugar alcohols, e.g., ISOMALT. These hard-boiled dosage forms were designed to be faster dissolving than prior lozenges described above. It has been found however that while these are pleasant, aesthetically appealing dosage forms, the rate of release of mixture is significantly greater than the rate of nicotine transferred across the buccal mucosa resulting in an undesired level of nicotine absorption in the GI tract.

A modified release hard-boiled oral dosage form for an active ingredient, e.g., nicotine, would be a useful addition to the art.

### SUMMARY OF THE INVENTION

In accordance with the present invention, a novel modified release, hard-boiled solid oral dosage form for an active ingredient, e.g., nicotine, is disclosed. The solid oral dosage form useful for transmucosal oral administration of an active, comprises:

a) a glassy matrix comprising at least one substantially non-hygroscopic sugar alcohol capable of forming a glassy structure;

b) a water soluble gelling gum in an amount sufficient to provide a desired oral dissolution rate of said glassy matrix; and

c) an active ingredient. Preferably, the active ingredient is a source of nicotine. The present invention further includes novel processes for preparing the present oral dosage forms and novel methods of nicotine replacement therapy using the present oral dosage forms when the active is nicotine.

### DRAWINGS

FIG. 1 shows the dissolution profile (% nicotine release vs time) comparing nicotine formulations without any gums to nicotine formulations according to the present invention.

### DETAILED DESCRIPTION OF THE INVENTION

All publications, including but not limited to patents and patent applications, cited in this specification are incorporated herein by reference as though fully set forth.

Unless otherwise specified, all parts and percentages set forth herein are weight percentages based on the weight of the relevant composition.

Unless otherwise stated, as used herein, the modifier “a” includes one or more of the components modified.

The present invention may comprise, consist essentially of, or consist of the components set forth below, unless otherwise stated. As mentioned above, copending application PCT/US02/08914 (C75119) discloses a hard boiled matrix for oral administration of nicotine suitable for use in a nicotine replacement therapy. That composition is preferably a mixture of substantially non-hygroscopic sugars, e.g., ISOMALT and nicotine in an amount sufficient to reduce nicotine cravings. This was designed to release nicotine more rapidly than prior art lozenges while providing a more pleasant, palatable dosage form expected to enhance compliance. Gums were avoided in these prior dosage forms since they tend to burn and tend to retain water during the hard-boil process steps. Unfortunately, such dosage forms released nicotine more rapidly than could be accomodated by the absorption rate across the buccal mucosa and an unacceptably high level of mixture was being absorbed in the gut.

In accordance with the present invention, it has now been found that small amounts of water soluble gelling gums can be added to such glassy matrix dosage forms without adversely impacting the hard-boiling process by which such forms are produced. The result is an oral dosage form which dissolves more slowly providing a greater degree of active, e.g., nicotine, being absorbed via oral mucosa.

Thus, the solid, oral dosage form of the present invention preferably comprises:

a) a glassy matrix comprising at least one substantially non-hygroscopic sugar alcohol capable of forming a glassy structure;

b) a water soluble gelling gum in an amount sufficient to provide a desired oral dissolution rate of the glassy matrix, and

c) an effective amount of an active.

The present oral dosage form can be used to deliver any active or therapeutic agent where absorption across the oral mucosa is desired. While the present invention will hereinafter be discussed with reference to administration of nicotine, it should be understood that other actives may be adjunctively or alternatively employed. Non-limiting examples include drugs, cough/cold/throat agents, vitamins, zinc, menthol, eucalyptus, hexyl resorcinol, caffeine, tooth whitening agents, anti-plaque agents, breath freshening agents, demulcents and the like.

The composition is orally dissolvable and may be in any form which is typically sucked, licked, and/or chewed and eaten, such as lozenges, sticks, canes, pops, etc. Lozenges are a preferred form. Lozenges of the present invention are oral dosage forms intended to be held in the mouth, and are typically sucked. For example, they may be held in the buccal cavity or sublingually. The lozenges may be in various shapes, including flat, circular, octagonal and biconvex.

The matrix (aka base) is a carrier for the nicotine active and optional adjuvants, and typically comprises from about 50% to about 100% of the composition. The product matrix is in a glassy, i.e., amorphous, physical state. Without intending to be limited or otherwise bound by theory, it is believed that the glassy matrix structure stabilizes nicotine actives such as nicotine and its derivatives, and potentially other components that tend to be unstable to moisture, e.g., by reducing penetration of water into the oral dosage form. The glassy matrix structure also tends to be more esthetically appealing to the user, e.g., providing a desirably smooth, organoleptic feel, which may increase user compliance. In addition, the glassy matrix structure tends to dissolve more rapidly than commercially available compressed nicotine tablets of which the present inventors are aware, thereby providing potentially faster craving relief than such tablets. However, by the addition of small amounts of water soluble gelling gums, the present dosage forms dissolve at a controlled rate so as to enhance the amount of nicotine absorbed orally rather than in the GI tract.

Glassy structure can be readily determined by those skilled in the art using conventional techniques such as X-ray diffraction. See, e.g., Settle, Frank A. et. al., Handbook of Instrumental Techniques for Analytical Chemistry, Prentice Hall PTR (1997). The formation of a glassy state is also typically characterized by a transparent appearance. As will be appreciated by those skilled in the art, the physical state is influenced by the properties of the components (especially sugar alcohols and other sugar components), and the process of making the product, and those skilled in the art will be able to select appropriate components and processes.

As mentioned above, the amount of water soluble gelling gum can be any convenient amount which produces the desired dissolution rate of the lozenge in the mouth while still maintaining the desired glassy matrix produced by the hard-boiled process. Preferably this includes about 0.5 to about 5% by weight of one or more water soluble gelling gums in the present oral dosage forms. More preferably, the present dosage forms include 1 to 4% and even more preferably 2.0 to 3.5% by weight of the gums.

Alternatively, the amount of water soluble gelling gums included can be viewed from a functional perspective in that glassy matrix dosage forms in accordance with the present invention contain sufficient gums to provide a dissolution rate which, in turn, results in at least 50% of the nicotine active being absorbed via the oral mucosa. More preferably, at least 70% and most preferably at least 80% of the nicotine active is absorbed across the oral mucosa as opposed to absorption in the GI tract from swallowing.

Any pharmaceutically acceptable water soluble gelling gums can be used in accordance with the present invention, as long as they can be incorporated into the hard-boil process described herein and provide the desired dissolution rate for the final product. Xanthan gum, guar gum, gum arabic and carrageenan are believed to be illustrative and particularly useful, with xanthan gum being preferred.

The non-hygroscopic property of the sugar alcohol is also believed to contribute to the stability of nicotine actives such as nicotine and its derivatives, and potentially other components that may be moisture-sensitive, as well as reducing the tendency of the oral dosage form to tackify upon exposure to humidity. As used herein, the term “substantially non-hygroscopic” means that the sugar alcohol has a low tendency to absorb water under conditions of 25° C./80% relative humidity (rh) (e.g., a maximum of 50%, preferably a maximum of about 30%, more preferably a maximum of about 20%, even more preferably a maximum of about 10% (e.g., up to about 8%), especially a maximum of about 5% (also up to about 2% or about 1%), weight gain of water upon exposure to conditions of 25° C./80% rh for a period of 2 weeks).

Examples of substantially non-hygroscopic sugar alcohols capable of forming a glassy structure suitable for use in the present invention include a sugar alcohol mixture comprising 1,6-GPS (6-O-α-D-glucopyranosyl-D-sorbitol) and 1,1-GPM (1-O-α-D-glucopyranosyl-D-mannitol) in a weight ratio of from about 1:99 to about 99:1, more preferably from about 70:30 to about 30:70, even more preferably from about 40:60 to about 60:40. In a particularly preferred embodiment the ratio is from about 43% to about 57% of 1,1-GPM and from about 57% to about 43% of 1,6-GPS (including about 1:1); for example, the sugar alcohol mixture contained in the product ISOMALT. ISOMALT is particularly preferred in the present invention. Such sugar alcohol mixtures may comprise other sugar alcohols and oligosaccharides, e.g., 1,1-GPS (1-O-α-D-glucopyranosyl-D-sorbitol), sorbitol, or mannitol, preferably in small amounts (e.g., less than about 10%, especially less than about 5%).

Sugar alcohol mixtures suitable for use in the invention are commercially available from Palatinit of America, Inc., of Morris Plains, N.J., USA. Suitable mixtures are also described in EP 0625578 B 1.

The substantially non-hygroscopic sugar alcohol serves as a carrier (or bulking agent) for the nicotine actives and optional adjuvants. The solid, oral dosage form typically comprises at least about 40% of the sugar alcohol, preferably at least about 50%, more preferably at least about 70%, most preferably at least about 85%, based on the weight of the dosage form.

As used herein, “nicotine active” refers to one or more compounds selected from nicotine, derivatives of nicotine such as salts and nicotine complexes, tobacco extract or leaf, and other pharmacologically active compounds which are useful for reducing cravings for nicotine, such as lobeline. As used herein, “cravings for nicotine” include cravings associated with tobacco usage, such as smoking and chewing tobacco.

A variety of nicotine actives are well known in the art and are commercially available. Specific examples of nicotine actives suitable for use in the present invention include nicotine oil, nicotine bitartrate, and nicotine complexed with cyclodextrin or polymer resins (e.g., nicotine polacrilex). Preferred nicotine actives are nicotine bitartrate, nicotine polacrilex, nicotine oil, and combinations thereof, especially nicotine bitartrate. The nicotine active may be used in one or more distinct physical forms well known in the art, including free base forms, encapsulated forms, ionized forms, and spray-dried forms.

The oral dosage form comprises one or more nicotine actives in an amount effective to reduce nicotine cravings, preferably within one hour of starting oral administration. In preferred embodiments, the product configuration, including the amount of nicotine active, is effective to reduce nicotine cravings either rapidly (e.g., within about 10 minutes, preferably within about 5 minutes), over a prolonged period (e.g., at least about 1 hour, preferably at least about 2 hours), or both, preferably both. Such combined rapid and prolonged craving relief may result from either the nicotine active per se or a combination of the nicotine active with other means which reduce acute or extended nicotine cravings (e.g., non-pharmacological sensory signals (including taste, tactile, scent signals} provided by inert components, such as flavor, cooling, tingling, effervescence). For example, the composition may comprise one or more flavors to provide rapid craving relief and an amount of nicotine active effective to provide relief of prolonged cravings.

In general, the amount of nicotine active may vary depending on the recommended or permitted therapeutic dosage for the particular nicotine active. Such dosages are known or ascertainable by conventional methods by those skilled in the medical arts. The composition preferably comprises from about 0.5 mg to about 5 mg of nicotine active per unit dosage form, more preferably from about 1 to about 4 mg nicotine active per unit dosage form.

The nicotine active is preferably substantially contained in the glassy matrix, and may be uniformly distributed throughout the matrix or distributed in one or more regions of the matrix.

The oral dosage form of the present invention may contain one or more optional ingredients, including ingredients such as are known in the art, e.g., buffers, flavorings, sugars, other sugar alcohols, high intensity sweeteners, colorants, vitamins, and antioxidants. Such optional ingredients may be used as adjuvants or as co-carriers for the nicotine active and optional components (e.g., sugars and sugar alcohols may be a co-carrier).

One or more buffer materials are especially desirable to facilitate transmucosal absorption of nicotine actives such as nicotine and nicotine derivatives. The buffer provides an alkaline mouth saliva pH that tends to enhance transmucosal absorption of such nicotine actives. Suitable buffer materials include inorganic or organic bases which have the capability to provide a mouth saliva pH of from above 7.0 to about 12.0, preferably above 7.0 to about 11.0, more preferably from about 7.5 to about 10.0, also about 7.5 to about 9.0. Suitable buffer materials include sodium carbonate, sodium bicarbonate, calcium carbonate, potassium carbonate, potassium bicarbonate, sodium phosphate dibasic, sodium phosphate tribasic, potassium phosphate dibasic and potassium phosphate tribasic. The buffer preferably comprises sodium carbonate, potassium carbonate, or a mixture thereof.

Preferably sufficient buffer is used such that the mouth saliva pH becomes and remains alkaline while the oral dosage form is held in the mouth during oral administration. When used, the composition generally comprises from about 0.2% to about 5.0% (e.g., about 0.5% to about 1.5%) buffer.

One or more sugars or other sugar alcohols may be used, e.g., as bulking agents. It has been found that such other sugar components may reduce the processing temperature required to form the oral dosage form, thereby tending to maintain stability of nicotine actives such as nicotine and its derivatives, and to increase the cost effectiveness of the process. Suitable other sugar components include sucrose, sorbitol, and xylitol, and in a preferred embodiment is sorbitol.

It is preferred that the oral dosage form is itself substantially non-hygroscopic and glassy. Therefore, the type and amount of optional other sugar components will preferably be selected such that the oral dosage form is substantially non-hygroscopic and glassy. In preferred embodiments, the oral dosage form absorbs a maximum of about 30% water by weight, more preferably a maximum of about 20% water by weight, even more preferably a maximum of about 10% by weight (e.g., up to about 8% by weight), still more preferably a maximum of about 5% by weight (especially a maximum of about 1-2% by weight), upon exposure to conditions of 25C/80% rh for a period of 2 weeks. Typically the oral dosage form will comprise from 0% to about 20%, e.g., from about 1% to about 20% or from about 10% to about 20% of such other sugar components, inclusive of any such components that may be present in the required sugar alcohol component. The composition may comprise higher levels of such other sugar components, provided that the matrix structure and hygroscopicity are acceptable.

High intensity sweeteners are useful for improving the sweetness profile of the composition, e.g., to provide a sweetness degree similar to table sugar. High intensity sweeteners are well known in the art and include soluble saccharin salts (e.g., sodium, calcium salts), the free acid form of saccharin, cyclamate salts, aspartame, Acesulfame-K (the potassium salt of 3,4-dihydro-6-methyl-1,2,3-oxathiazine-4-one-2,2-dioxide), and sodium, ammonium, or calcium salts of 3,4-dihydro-6-methyl-1,2,3-oxathiazine-4-one-2,2-dioxide. Preferred high intensity sweeteners are Acesulfame-K and aspartame, especially Acesulfame K. High intensity sweeteners, when used, typically comprise from about 0.001% to about 5% of the composition, more typically up to about 0.5% by weight of the composition.

Flavoring agents may be any natural or synthetic flavors such as known in the art, including mints (e.g., peppermint, spearmint), menthol, citrus (e.g., orange, lemon), other fruit flavors, vanilla, cinnamon, chocolate, coffee and tobacco flavor. When used, the composition typically comprises a total of from about 0.25 to about 5 weight % of one or more flavorings.

Colorants include pigments, natural food colors and dyes which are suitable for food and drug applications, e.g., F.D.C. dyes and lakes. Colorants typically comprise from about 0.001% to about 0.05% of the composition.

Vitamins such as vitamin C and E may be included.

Small amounts of vegetable oils, e.g., sesame oil, may be added to the composition as a processing aid, more particularly as an anti-adhesive agent/lubricant to prevent the composition from sticking to equipment, molds, and the like. Typically up to about 1% of such oils, based on the weight of the composition, may be used.

Small amounts of citric acid may be included, e.g., to prevent discoloration of the composition during processing.

The solid oral dosage forms may also contain pharmaceutically acceptable polymers and binders and/or mixtures of such polymers and binders. Such polymers ad binders include, but are not limited to:

Homo- or copolymers of N-vinylpyrrolidone such as polyvinylpyrrolidone (PVP), copolymers of N-vinylpyrrolidone with vinylesters, especially with vinylacetate, or also with vinylpropionate. Copolymers of vinylacetate and crotonic acid, partly saponified polyvinylacetate or polyvinylalcohol;

Cellulose derivatives such as, cellulose ether, especially methyl cellulose, ethyl cellulose, hydroxyalkyl celluloses, especially hydroxypropyl cellulose, hydroxyalkyl alkyl celluloses, especially hydroxypropyl methyl cellulose and hydroxypropyl ethyl cellulose. Cellulose esters such as cellulose phthalate;

Also suitable as polymer binders are polymers with an acrylate or methacrylate base, for example the polyacrylates and polymethacrylates, copolymers of acrylic acid and methylmethacrylate or polyhydroxyalkyl acrylates or methcrylates;

Also suitable are polyactides, polyglycolides, polyactide-polyglycolides, polydioxans, polyanhydrides, polystyrene sulfonates, polyacetates, polycaprolactones, poly(ortho)esters, polyamines, polyhydroxyalkanoates or alginates;

Suitable matrix components may also be natural or semi-synthetic binders such as starches, decomposed starches, for example maltodextrine, as well as gelatin which may have a basic or acidic character as required, chitin or chitosan.

Mixtures of polymers and binders may be used. Especially preferred mixtures include thermoplastically processable polymers with Isomalt.

The solid, oral dosage forms may be suitably prepared by methods known in the art of hard confectionaries, e.g., hard-boiled confectionaries. A general discussion of preparation of hard confectionaries may be found in H. A. Lieberman, Pharmaceutical Dosage Forms: Tablets, Vol. 1 (1980), Marcel Dekker, Inc., NY, N.Y., especially pp. 339-469. Particular apparatus for making the oral dosage form includes cooking and mixing apparatus known in the confectionary manufacturing arts, and appropriate apparatus will be apparent to the skilled artisan.

In general, preparation of the solid, oral dosage form involves:

(1) with mixing and heating, forming a melt of the substantially non-hygroscopic sugar alcohol and optionally, other sugar components and/or a diluent such as water;

(2) cooking the melt;

(3) removing excess moisture from the melt (e.g., to less than about 2% moisture);

(4) cooling the melt with mixing until the melt is a plastic-like, workable mass;

(5) while the melt is a plastic-like mass, incorporating the nicotine active, the one or more water soluble gelling gums and any remaining optional ingredients; and

(6) forming the plastic-like mixture into solid, oral dosage forms having the desired size and shape.

Methods known in the art of making hard confectioneries include those utilizing fire cookers, vacuum cookers, and scraped-surface cookers (aka high speed atmospheric cookers).

For example, in one suitable fire cooker method, the desired quantity of the substantially non-hygroscopic sugar alcohol and any other sugar components are dissolved in water by heating them in a kettle until dissolved. Additional sugar components may be added and cooking continued until a final temperature of about 145-165° C. is achieved. The mix is then cooled, worked as a plastic-like mass, and admixed with the nicotine active, gum and optional ingredients such as flavors, colorants, buffer, etc.

In a suitable vacuum cooker method, the sugar components are boiled at a temperature of about 125-132° C., vacuum is applied and additional water is boiled off without extra heating. When cooking is complete, the mass is a semi-solid having a plastic-like consistency. The nicotine active, gum and any optional ingredients are admixed into the mass at this point by conventional methods.

In a suitable method using scraped-surface cookers, a film of a mixture of the sugar components is spread on a heat exchange surface and heated to about 165-170° C. within a few minutes. The composition is then rapidly cooled to about 100-120° C. and worked as a plastic-like mass, mixing in the nicotine active, gum and any optional ingredients.

In the foregoing methods, the cooking temperature should be sufficiently high to drive water from the mix. Where vacuum is employed, lower temperatures can typically be used. In order to avoid discoloration of the sugar components, the buffer may be added at a temperature below 130° C., e.g. from 80° C. to 130° C., more preferably between 120° C. and 125° C. In order to facilitate formation of a transparent product, the buffer is preferably added as a solution. The nicotine active is preferably added as a preblend comprising a sugar or sugar alcohol component, to help ensure uniform dosage. The one or more gums can be incorporated into this preblend or added alone. The ingredients are mixed for a period to provide a homogeneous mixture, typically from about 4 to about 10 minutes. Once the composition has been properly tempered, it may be cut into workable portions or otherwise formed into desired shapes and sizes using forming techniques such as are known in the art.

The process of preparation can be adapted by those skilled in the art to provide solid dosage forms having a desired configuration, including single-layer, multi-layer having two or more layers (e.g., 3 layers), and forms having a center core. For example, the nicotine active may be distributed in one or more layers, in a portion of a layer, included in a center core (e.g., surrounded wholly or in part by another composition, preferably comprising the glassy matrix), or otherwise concentrated in one or more regions of the oral dosage form.

In preferred embodiments the oral dosage form is configured such that the buffer and nicotine active are substantially separated, e.g., to reduce the potential for reaction between the active and buffer. Such embodiments are preferably configured to facilitate transmucosal absorption of the nicotine active, e.g., such that the buffer and nicotine active are released approximately simultaneously. For example, the buffer and nicotine may be present in separate outer layers of the oral dosage form, optionally with one or more other layers sandwiched there between. Such sandwich layers are preferably inert to the buffer and nicotine active. Alternatively, either the buffer or nicotine active may be present in a center core, with the other component, respectively, being present in a composition, preferably comprising the glassy matrix, surrounding the core wholly or in part (e.g., in an outer ring encircling the core). In another embodiment, the buffer may be included in a portion of a layer, with the nicotine active being included in another, separate portion of the layer (e.g., half buffer, half active).

The oral dosage form of the present invention is useful as a tobacco replacement, and as a means to reduce or stop tobacco use, including smoking tobacco (cigarettes, pipe tobacco, cigars), and chewing tobacco. The oral dosage form may be used as a total or partial replacement of tobacco, and can be used concurrently with tobacco in a planned tobacco reduction program (e.g., while reducing tobacco usage prior to quitting tobacco usage).

Therefore, the present invention also relates to a method of reducing tobacco usage, comprising orally administering a solid, oral dosage form of the present invention to a person in need of such reduction. The present invention also relates to a method of reducing nicotine cravings comprising orally administering a solid, oral dosage form of the present invention to a person in need of nicotine craving reduction. “Need” is intended to include a person's desire to reduce tobacco usage or nicotine cravings, respectively. Reducing nicotine cravings or tobacco usage includes stopping nicotine cravings or tobacco usage, respectively.

In general, in these methods the oral dosage form is administered as needed to prevent or reduce nicotine cravings, within any recommended or permitted limits. The oral dosage form is typically administered such that the nicotine active is primarily delivered transmucosally in the mouth. Useful regimens may include those which provide a sustained nicotine blood plasma concentration of from about 6 ng/ml to about 35 ng/ml. Fast craving relief may be perceived by users where, for example, the composition is configured to provide a nicotine blood plasma concentration of at least about 6 ng/ml, especially at least about 12 ng/ml, within about 10 minutes of starting administration, especially within about 5 minutes of starting administration.

For example, for lozenge forms, up to about 15 lozenges comprising 4 mg nicotine or its equivalent may be used per day. The number of lozenges used per day may be adjusted upward or downward for lower or higher unit dosage strengths, respectively, to provide equivalent regimens.

### EXAMPLES

Without further elaboration, it is believed that one skilled in the art can, using the preceding description, utilize the present invention to its fullest extent. The following Examples, therefore, are to be construed as merely illustrative and not a limitation of the scope of the present invention.

### Examples 1-9

Nine batches of lozenges in accordance with the present invention were prepared as set forth below.

600 Kg of Isomalt powder type M was dissolved in 300 Kg of water and cooked to a temperature of 160° C. and then placed under vacuum for three minutes in a Hamac-Hansella candy processor to form a molten base. Thereafter when the bolten base had cooled to about 125° C., the nicotine polacrilex, Xanthan Gum, Acesulfame K, Sodium Carbonate and flavor were mixed in. The so-formed mixture was transferred to a Ruffinatti kneading table and kneaded. Thereafter the mixture was formed, spun and sized into a rope from which lozenges were punched using a Bosch Uniplast.

| | | Amount Per Examnle* | | | | | | | | |
|---|---|---|---|---|---|---|---|---|---|---|
| Ingredient | | 1 | 2 | 3 | 4 | 5 | 6 | 7 | 8 | 9 |
| Isomalt M Cooked Base | | 33.9 | 33.4 | 33.0 | 33.8 | 33.2 | 32.9 | 33.9 | 33.4 | 33.0 |
| Nicotine Polacrilex | | 0.324 | 0.324 | 0.324 | 0.324 | 0.324 | 0.324 | 0.324 | 0.324 | 0.324 |
| Sodium Carbonate | | 0.27 | 0.27 | 0.27 | 0.27 | 0.27 | 0.27 | 0.27 | 0.27 | 0.27 |
| | kg | 0.35 | 0.88 | 1.23 | 0.35 | 0.88 | 1.23 | 0.35 | 0.88 | 1.23 |
| Xanthan Gum | % w/w | 1.0% | 2.5% | 3.5% | 1.0% | 2.5% | 3.5% | 1.0% | 2.5% | 3.5% |
| Acesulfame K | | 0.042 | 0.042 | 0.042 | 0.042 | 0.042 | 0.042 | 0.042 | 0.042 | 0.042 |
| Cappuccino Flavor | | 0.105 | 0.105 | 1.105 | | | | | | |
| Anise Mint Flavor | | | | | 0.050 | 0.050 | 0.050 | | | |
| Natural Masking Agent | | | | | 0.206 | 0.206 | 0.206 | | | |
| Chai Spice Flavor | | | | | | | | 0.126 | 0.126 | 0.126 |

*(Amounts are “After Processing” in Kg)

### Example 10

The dissolution profiles were measured for nicotine lozenges containing 3.5% xanthan gum per Example 3 above, for nicotine lozenges per Example 3 but containing 5.0% xanthan gum, and for a non-gelling gum formulation (similar to Example 3 herein, but with no gum) of the aforementioned copending PCT International Application No. PCT/US02/08914, filed Mar. 22, 2002.

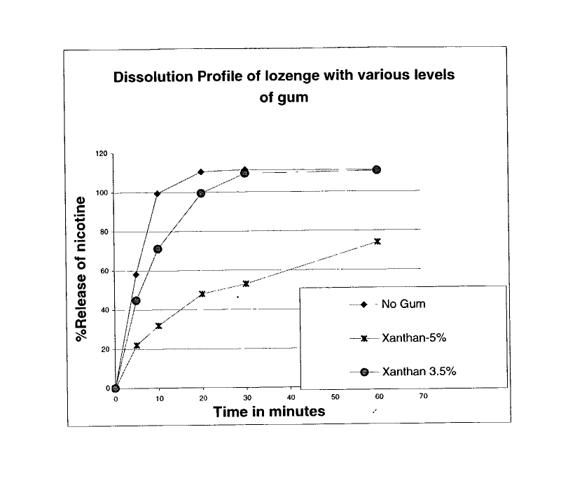

The dissolution profile is determined by using Standard USP 3 Bio.Dis., dipping the lozenges at a standard rate in a solution buffered to pH 7.4 to mimic conditions in the oral cavity. The chart of FIG. 1 plots percent release of nicotine by assay versus time in minutes.

What is claimed is:

1. A solid, oral dosage form useful for transmucosal oral administration of an active agent, comprising:
a) a glassy matrix comprising at least one substantially non-hygroscopic sugar alcohol capable of forming a glassy structure;
b) a water soluble gelling gum in an amount sufficient to provide a desired oral dissolution rate of said glassy matrix; and
c) said active agent.

2. The dosage form of claim 1 wherein said active agent is one or more selected from the group consisting of drugs, cold agents, cough agents, throat agents, vitamins, zinc, menthol, eucalyptus, hexylresorcinol, caffeine, tooth whitening agents, anti-plaque agents, breath freshening agents and nicotine.

3. The dosage form of claim 2 wherein said active is nicotine.

4. The dosage form of claim 1 wherein said water soluble gelling gum is one or more selected from the group consisting of xanthan gum, guar gum, gum arabic, and carageenan.

5. The dosage form of claim 4 wherein said gum is xanthan gum.

6. The dosage form of claim 3 wherein said dissolution rate is sufficient to provide that at least 50% of said nicotine is delivered via the oral mucosa prior to ingestion into the stomach.

7. The dosage form of claim 6 wherein at least 75% of said nicotine is delivered via the oral mucosa.

8. The dosage form of claim 1 wherein said gum is present in an amount sufficient to provide that said form dissolves orally over a period of about 10 to 15 minutes.

9. The dosage form of claim 1 wherein said gum is present in an amount of from about 0.5 to about 5.0 percent by weight.

10. The dosage form of claim 9 wherein said gum is present in an amount of from about 1.0 to about 4.0 percent by weight.

11. The dosage form of claim 10 wherein said gum is present in an amount of from about 1.0 to 3.5 percent by weight.

12. A dosage form of claim 1 wherein the sugar alcohol comprises a mixture of 1,6-GPS (6-O-α-D-glucopyranosyl-D-sorbitol) and 1,1-GPM (1-O-α-D-glucopyranosyl-D-mannitol) in a weight ratio of from about 99:1 to about 1:99.

13. A dosage form of claim 1 comprising at least about 50% of the sugar alcohol, based on the weight of the dosage form.

14. A dosage form of claim 13 comprising at least about 70% of the sugar alcohol mixture, based on the weight of the dosage form.

15. A dosage form of claim 14 comprising at least about 85% of the sugar alcohol mixture, based on the weight of the dosage form.

16. A dosage form of claim 1 wherein the nicotine active is selected from nicotine, derivatives of nicotine, and combinations thereof.

17. A dosage form of claim 16 wherein the nicotine active is selected from nicotine oil, nicotine bitartrate, nicotine polacrilex and combinations thereof.

18. A dosage form of claim 1 comprising from about 0.5 mg to about 5 mg of the nicotine active per dosage unit.

19. A dosage form of claim 12 wherein the sugar alcohol comprises a mixture of 1,6-GPS and 1,1-GPM in a weight ratio of from about 70:30 to about 30:70.

20. A dosage form of claim 12 wherein the sugar alcohol comprises a mixture of 1,6-GPS and 1,1-GPM in a weight ratio of from about 60:40 to about 40:60.

21. A dosage form of claim 12 wherein the sugar alcohol mixture is ISOMALT.

22. A dosage form of claim 1 further comprising a buffer in an amount effective to provide an alkaline mouth saliva pH.

23. A dosage form of claim 22 wherein the buffer is selected from sodium carbonate, sodium bicarbonate, calcium carbonate, potassium carbonate, potassium bicarbonate, sodium phosphate dibasic, sodium phosphate tribasic, potassium phosphate dibasic, potassium phosphate tribasic, and combinations thereof.

24. A dosage form of claim 23 wherein the buffer is selected from sodium carbonate, potassium carbonate, and combinations thereof.

25. A dosage form of claim 1 wherein the glassy matrix further comprises from about 1% to about 20%, based on the weight of the dosage form, of one or more compounds selected from the group consisting of sucrose, sorbitol, and xylitol.

26. A dosage form of claim 3 further comprising a non-pharmacological component for providing a sensory signal effective to provide rapid nicotine craving relief.

27. A dosage form of claim 3 in the form of a lozenge.

28. A method of reducing nicotine cravings comprising orally administering a dosage form of claim 3 to a person in need of nicotine craving reduction.

29. A method of claim 28 wherein a nicotine active blood plasma concentration of at least about 6 ng/ml is achieved after starting oral administration of the dosage form.

30. A method of claim 29 wherein a sustained nicotine active blood plasma concentration of from about 6 ng/ml to about 35 ng/ml is achieved after starting oral administration of the composition.

31. A method of reducing tobacco usage comprising orally administering a dosage form of claim 3 to a person in need of reducing tobacco usage.

32. A solid, oral dosage form useful for transmucosal oral administration of a nicotine active, wherein the dosage form provides a nicotine active blood plasma concentration of at least about 6 ng/ml after starting oral administration of the dosage form.

33. A solid, oral dosage form useful for tansmucosal oral administration of a nicotine active, wherein the dosage form provides a sustained nicotine active blood plasma concentration of from about 6 ng/ml to about 35 ng/ml after starting oral administration of the dosage form.
